(19) United States
(12) Patent Application Publication
(10) Pub. No.: US 2021/0212661 A1
(43) Pub. Date: Jul. 15, 2021

(54) SYSTEM AND METHOD FOR MEDICAL ULTRASOUND WITH MONITORING PAD

(71) Applicant: Sonoscope Inc., Longueuil (CA)

(72) Inventors: Maxime VALOIS, Longueuil (CA); Jean-François LANCTÔT, Longueuil (CA); Hugo DOUVILLE, Longueuil (CA); Yannick LE DEVEHAT, Quebec City (CA); Bernard LEFEBVRE, Boischatel (CA)

(21) Appl. No.: 17/215,957

(22) Filed: Mar. 29, 2021

Related U.S. Application Data
(63) Continuation of application No. PCT/CA2020/051108, filed on Aug. 13, 2020.
(60) Provisional application No. 62/886,638, filed on Aug. 14, 2019.

(51) Int. Cl. A61B 8/00; A61B 8/02; A61N 1/365; A61N 1/04; A61N 1/08; B06B 1/06

(52) U.S. Cl. CPC A61B 8/4281; A61B 8/4494; A61B 8/461; A61B 8/4477; B06B 1/0622; A61N 1/36528; A61N 1/0492; A61N 1/08; A61B 8/02

(57) ABSTRACT

Disclosed is an ultrasound system having a monitoring pad for application to a patient, an ultrasound probe that connects to the monitoring pad and has a plurality of ultrasound transducers, and an ultrasound beamforming device configured to control the ultrasound transducers to focus an ultrasound beam into the patient and to read resulting reflections of the ultrasound beam. The monitoring pad has an ultrasound gel pad and a support structure that holds the ultrasound gel pad. In accordance with an embodiment of the disclosure, the support structure is geometrically configured to receive the ultrasound probe and to hold it in a fixed arrangement against the ultrasound gel pad, such that the ultrasound gel pad is sandwiched between the patient and the ultrasound transducers. In some implementations, the monitoring pad has electrocardiogram electrodes and/or other sensor(s) unrelated to ultrasound, and the ultrasound beamforming device receives readings from the same.

### RELATED APPLICATION

This patent application is a bypass continuation of PCT Patent Application No. PCT/CA2020/051108 filed on Aug. 13, 2020, which claims priority to U.S. Provisional Patent Application No. 62/886,638 filed on Aug. 14, 2019. Both the PCT Patent Application and the Provisional Patent Application are incorporated by reference in their entirety.

### FIELD OF THE DISCLOSURE

This disclosure relates to medical ultrasound, and more particularly to POCUS (Point-of-Care Ultrasound) and monitoring.

### BACKGROUND

Medical ultrasound (also known as diagnostic sonography or ultrasonography) is used to create an ultrasonic image of internal body structures such as tendons, muscles, joints, blood vessels, and internal organs. Ultrasonic images, also known as sonograms, are made by sending ultrasound pulses into a patient using a probe positioned on the patient, recording resulting reflections, and displaying an ultrasonic image based on the resulting reflections. Different tissues have different reflection properties, and thus different tissues can be distinguished in an ultrasonic image.

A medical ultrasound procedure normally involves a medical professional holding and manipulating the probe to obtain ultrasonic images of an area of interest. A gel is normally placed between the patient and the probe to facilitate travel of the ultrasound pulses into the patient and the resulting reflections back into the probe for recording. The gel can also help to facilitate the medical professional to manipulate the probe on the patient.

Unfortunately, the gel can be messy and can prompt a clean-up of both the patient and the probe, especially since movement of the probe smears the gel over a relatively large surface of the patient. Also, the probe can become contaminated by the patient, especially with movement of the probe against the patient. Therefore, the probe should be cleaned after each use, for example using soap and water, or quaternary ammonium sprays or wipes. This can be inconvenient and cumbersome.

POCUS (Point-of-Care Ultrasound) enables a medical ultrasound procedure to be performed on a patient wherever the patient is being treated, whether in a modern hospital, an ambulance, or a remote village. POCUS can improve patient care for very sick patients by providing sonographic information to medical professionals during emergency procedures such as cardiac resuscitation for example. POCUS can also improve patient care for other patients such as pregnant women having routine checkups for example.

Unfortunately, POCUS relies on the medical professional to hold and manipulate the probe using their professional skill. In some situations, such as cardiac arrest, this may not be practical or possible. For example, it is the standard of care for cardiac arrest patients worldwide to be monitored with a defibrillator device during cardiac resuscitation. Although defibrillators typically provide electric monitoring, i.e. heart rate and rhythm, they do not provide sonographic information. Thus, when using the defibrillator, there may be no sonographic information for the medical professional.

Moreover, image generation with POCUS can sometimes be challenging and induce delays in decision making, diagnosis, or patient care. In critical situations, such as cardiac resuscitation, these delays can be prohibitive to the use of POCUS despite the fact that POCUS could bring critical information. For example, POCUS can provide direct information on cardiac contractility, an information much more reliable than manual pulse check, the current standard of care in cardiac resuscitation.

Therefore, while POCUS can improve patient care, it leaves much to be desired. It is desirable to improve upon POCUS to address or mitigate some or all of the aforementioned shortcomings.

### SUMMARY OF THE DISCLOSURE

Disclosed is an ultrasound system having a monitoring pad for application to a patient, an ultrasound probe that connects to the monitoring pad and has a plurality of ultrasound transducers, and an ultrasound beamforming device configured to control the ultrasound transducers to focus an ultrasound beam into the patient and to read resulting reflections of the ultrasound beam. In some implementations, the ultrasound beamforming device uses 3D beam scanning algorithms to accomplish beamforming via the ultrasound transducers. The beamforming enables a medical ultrasound procedure to be accomplished without holding or manipulating the ultrasound probe or the monitoring pad, which can remain fixed on the patient. This improves upon conventional approaches in which an ultrasound transducer is held and manipulated by a medical professional using their professional skill.

The monitoring pad that is applied to the patient has an ultrasound gel pad and a support structure that holds the ultrasound gel pad. In accordance with an embodiment of the disclosure, the support structure is geometrically configured to receive the ultrasound probe and to hold the ultrasound probe in a fixed arrangement against the ultrasound gel pad. The ultrasound gel pad is sandwiched between the patient (i.e. the patient's skin) and the ultrasound probe, and serves as an ultrasound interface between the patient and the ultrasound probe without the ultrasound gel pad being smeared over a surface of the patient. This can improve upon conventional approaches by reducing an amount of clean-up after the medical ultrasound is performed. In some implementations, the monitoring pad is designed to be disposable after a single use or after a limited number of uses, which can help to reduce clean-up after the medical ultrasound and can help to ensure sanitary conditions.

Also disclosed is an ultrasound beamforming device configured to control an ultrasound transducer array with beamforming to acquire ultrasound data, to receive a reading from at least one sensor unrelated to ultrasound (e.g. electrocardiogram electrodes), and to concurrently display an ultrasound image based on the ultrasound data and another image (e.g. electrocardiogram) based on the reading from the other sensor. In this way, patient monitoring of heart and/or lung functions is possible, which can be of great value in resuscitation bays, operating rooms, critical care units, neonatology units and prehospital settings. This improves upon conventional approaches in which ultrasound systems rely on the medical professional to hold and manipulate the probe using their professional skill and hence are not suitable for monitoring patients.

Also disclosed is a method that involves applying the monitoring pad to a patient, connecting the ultrasound probe having the ultrasound transducers to the monitoring pad, and operating the ultrasound beamforming device to control the ultrasound transducers to focus an ultrasound beam into the patient and to read resulting reflections of the ultrasound beam. Notably, the ultrasound beamforming device can be operated without holding or manipulating the monitoring pad or the ultrasound probe. Again, this improves upon conventional approaches for similar reasons described above.

Other aspects and features of the present disclosure will become apparent, to those ordinarily skilled in the art, upon review of the following description of the various embodiments of the disclosure.

### BRIEF DESCRIPTION OF THE DRAWINGS

Embodiments will now be described with reference to the attached drawings in which:

FIG. 1 is a schematic of an ultrasound system having a monitoring pad, an ultrasound probe having a plurality of ultrasound transducers, and an ultrasound beamforming device;

FIG. 2 is a schematic of the monitoring pad on a patient;

FIG. 3 is a schematic of an exploded view of the ultrasound probe along with an exploded view of the monitoring pad;

FIG. 4 is a detailed view of a mechanism of the monitoring pad for receiving and holding the ultrasound probe;

FIG. 5A to FIG. 5C are schematics depicting the ultrasound probe connected to the monitoring pad;

FIG. 6A and FIG. 6B are schematics of an ultrasound transducer array of the ultrasound probe;

FIG. 7 is a block diagram of the ultrasound beamforming device operatively coupled to the ultrasound transducer array and another sensor unrelated to ultrasound;

FIG. 8 is a schematic of example information that can be displayed by the ultrasound beamforming device;

FIG. 9 is a schematic of a patient showing example placement of a monitoring pad between defibrillation pads; and

FIG. 10 is a flowchart of a method of using the ultrasound system.

### DETAILED DESCRIPTION OF EMBODIMENTS

It should be understood at the outset that although illustrative implementations of one or more embodiments of the present disclosure are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

### Ultrasound System

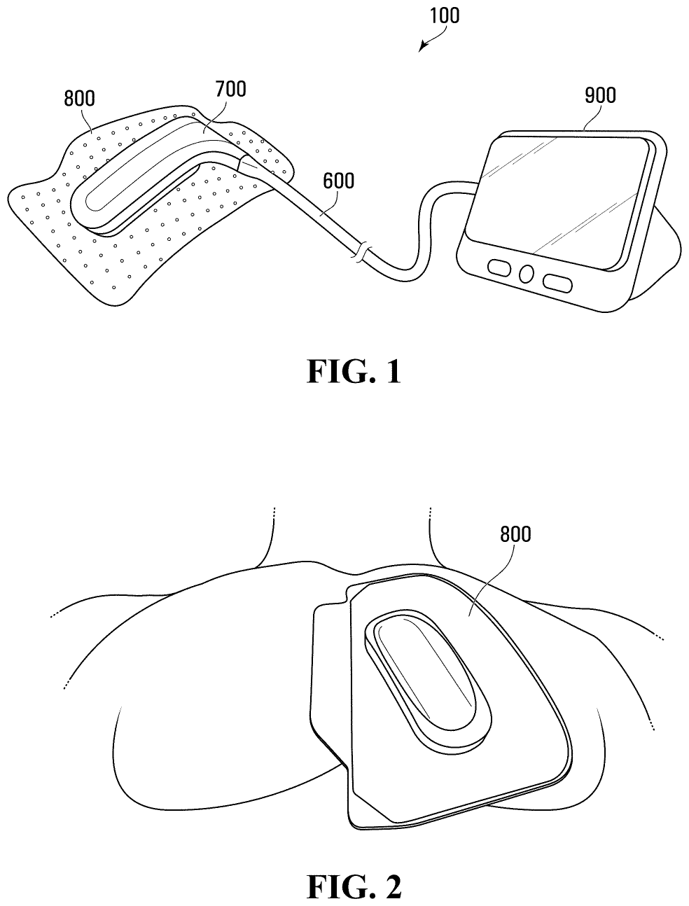

Referring first to FIG. 1, shown is a schematic of an ultrasound system 100. The ultrasound system 100 has a monitoring pad 800 for application to a patient, an ultrasound probe 700 that connects to the monitoring pad 800 and has a plurality of ultrasound transducers (not shown), and an ultrasound beamforming device 900 configured to control the ultrasound transducers to focus an ultrasound beam into the patient and to read resulting reflections of the ultrasound beam. The ultrasound beamforming device 900 is coupled to the ultrasound probe 700 via a cable 600, but could be coupled wirelessly in other implementations.

Operation of the ultrasound system 100 will now be described by way of example. The monitoring pad 800 can be applied to a patient. See for example FIG. 2, which shows a schematic of the monitoring pad 800 on a patient. Although the monitoring pad 800 is shown to be applied to the patient on their chest, it will be appreciated that monitoring pad 800 can be applied to any suitable location on the patient. In some implementations, as described in further detail below, the monitoring pad 800 has an adhesive layer for securing the monitoring pad 800 to the patient. However, other securing means are possible such as straps or bands, for example.

Referring back to FIG. 1, the ultrasound probe 700 connects to the monitoring pad 800, which is applied to the patient. During operation of the ultrasound system 100, the ultrasound beamforming device 900 controls the ultrasound transducers of the ultrasound probe 700 to send ultrasound pulses into the patient and to record resulting reflections. In some implementations, the ultrasound system 100 displays an ultrasonic image based on the resulting reflections. Different tissues have different reflection properties, and thus different tissues can be distinguished in the ultrasonic image. In some implementations, the ultrasound beamforming device 900 uses 3D beam scanning algorithms to accomplish beamforming via the ultrasound transducers. The beamforming enables an ultrasound beam to be focused into the patient. In this way, the ultrasonic image can be produced for an area of interest without holding or manipulating the ultrasound probe 700 or the monitoring pad 800, which can remain fixed on the patient. This improves upon conventional approaches in which an ultrasound transducer is held and manipulated by a medical professional using their professional skill.

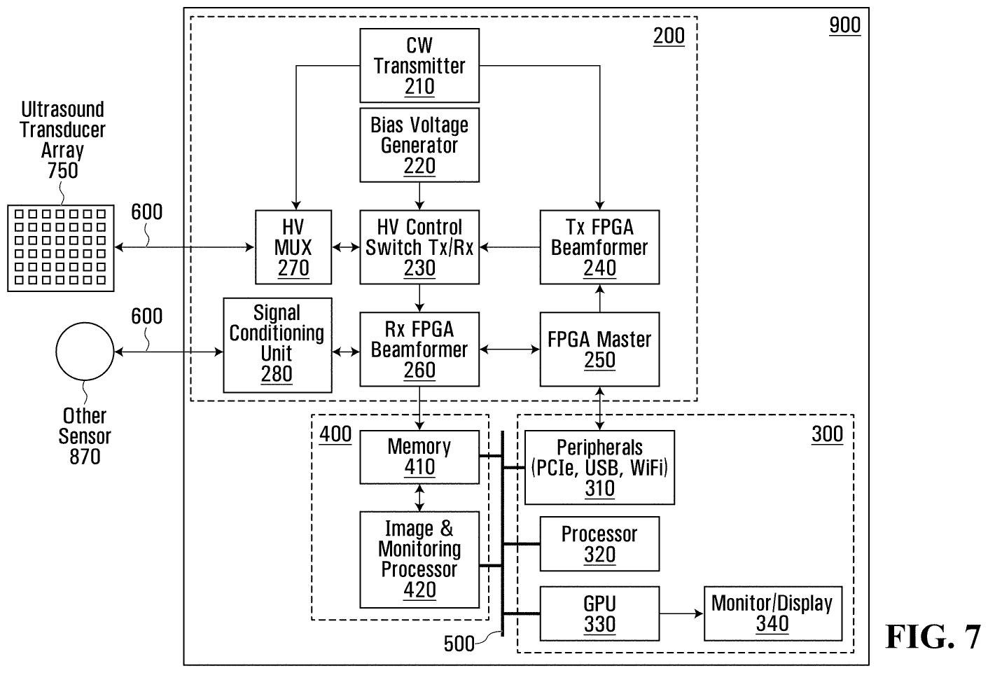

In some implementations, the ultrasound beamforming device 900 has transmission circuitry (not shown) to control a time-delay for exciting each ultrasound transducer in the ultrasound probe 700 to generate a plurality of ultrasound beams transmitted into the patient such that ultrasound energy is in phase at a predefined focal point within the patient, and the ultrasound beamforming device 900 has reception circuitry (not shown) to read resulting reflections of the ultrasound beam from the predefined focal point. In some implementations, the ultrasound beamforming device 900 is configured to refocus the plurality of ultrasound beams at a specific region of interest to improve signal to noise ratio. Example details of the transmission circuitry and the reception circuitry are provided later with reference to FIG. 7.

In some implementations, the ultrasound beamforming device 900 has a display for displaying an ultrasound image based on the resulting reflections of the ultrasound beam. In some implementations, to assist with physician diagnosis, the ultrasound beamforming device 900 implements pattern recognition or artificial intelligence to automatically generate a morphology or tissue identification (e.g. a specific plane of cut) based on the resulting reflections of the ultrasound beam. As a specific example, a multi-layer artificial neural network can be trained with training data to recognise patterns corresponding to target morphology or tissue identification, and then the multi-layer artificial neural network used to automatically generate a morphology or tissue identification for situations that are similar to those represented by the training data. However, other artificial intelligence methods such as machine learning decision tree algorithm may be used for pattern recognition and morphology identification, for example. Further example algorithms that can be implemented by the ultrasound beamforming device 900 are provided later with reference to FIG. 7.

### Monitoring Pad

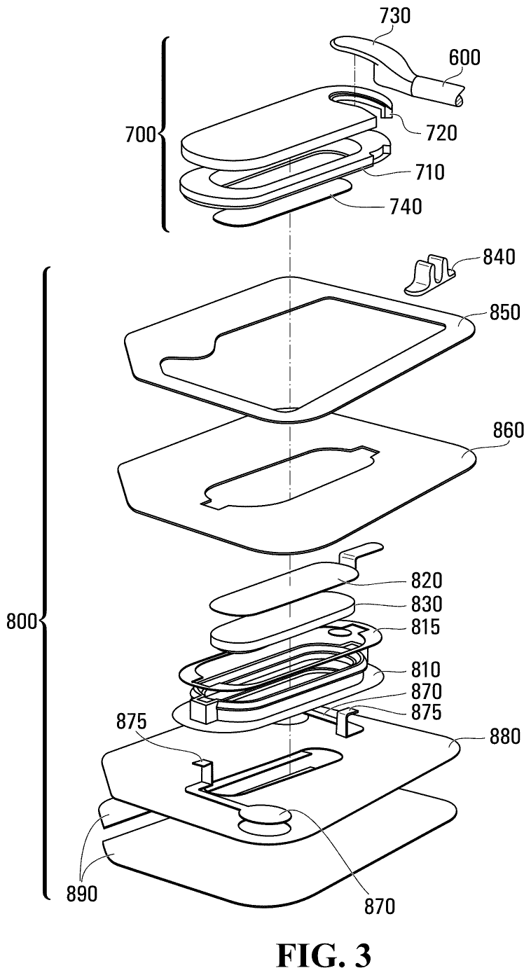

Referring now to FIG. 3, shown is a schematic of an exploded view of the ultrasound probe 700 along with an exploded view of the monitoring pad 800. The monitoring pad 800 has an ultrasound gel pad 830 and a support structure 810,840,850,860 that holds the ultrasound gel pad 830. In accordance with an embodiment of the disclosure, the support structure 810,840,850,860 is geometrically configured to receive the ultrasound probe 700 and to hold it in a fixed arrangement against the ultrasound gel pad 830, such that the ultrasound gel pad 830 is sandwiched between the patient (i.e. the patient's skin) and the ultrasound probe 700. In this way, the ultrasound gel pad 830 can serve as an ultrasound interface between the patient and the ultrasound transducers of the ultrasound probe 700. Notably, the ultrasound gel pad 830 involves little or no manipulation to provide a good ultrasound interface. Also, the ultrasound gel pad 830 does not cause a mess as in conventional approaches because the ultrasound gel pad 830 is generally contained by the monitoring pad 800 and is not smeared onto a surface of the patient. As a result, an amount of clean-up after the medical ultrasound is performed may be reduced compared to conventional approaches. In some implementations, the monitoring pad 800 is designed to be disposable after a single use or after a limited number of uses, which can further help to reduce clean-up after the medical ultrasound.

There are many possibilities for the support structure 810,840,850,860. In some implementations, the support structure 810,840,850,860 has a cradle 810 that holds the ultrasound gel pad 830 and is configured to receive the ultrasound probe 700 and hold the ultrasound probe 700 in the fixed arrangement, such that the ultrasound gel pad 830 is sandwiched between the patient and the ultrasound probe 700. In some implementations, the fixed arrangement provides for a continuous pressure between a surface of the ultrasound probe 700 and the ultrasound gel pad 830. The continuous pressure helps to enable the ultrasound gel pad 830 to serve as an ultrasound interface between the patient and the ultrasound transducers of the ultrasound probe 700, as air pockets are eliminated or reduced.

In the illustrated example, the cradle 810 is shown with a stadium shape for retaining the ultrasound gel pad 830. However, it is to be understood that other shapes are possible, for example an oval shape or a rectangular shape. Any suitable shape that retains the ultrasound gel pad 830 can be implemented. In general, the cradle 810 is geometrically designed such that the ultrasound gel pad 830 can be inserted and fixed.

In some implementations, the support structure 810,840,850,860 has a support layer 860,850 and a clip 840 coupled to the support layer 860,850. In some implementations, the support layer 860,850 has a backing layer 860 and a frame 850 for structural support, and the clip 840 and is configured to retain the cable 600 of the ultrasound probe 700 to the frame 850 of the support layer 860,850. In other implementations, the frame 850 is omitted when rigidity of the backing layer 860 sufficient for structural support.

The combination of the cradle 810, the support layer 860,850, and the clip 840 enable the ultrasound probe 700 to be secured to the ultrasound pad 800. In some implementations, the support structure 810,840,850,860 includes at least the cradle 810, the support layer 860,850, and the clip 840. In some implementations, the support structure 810,840,850,860 includes additional components, for example an adhesive layer 815 that bonds the cradle 810 to the bonded to the support layer 860,850. Other implementations are possible.

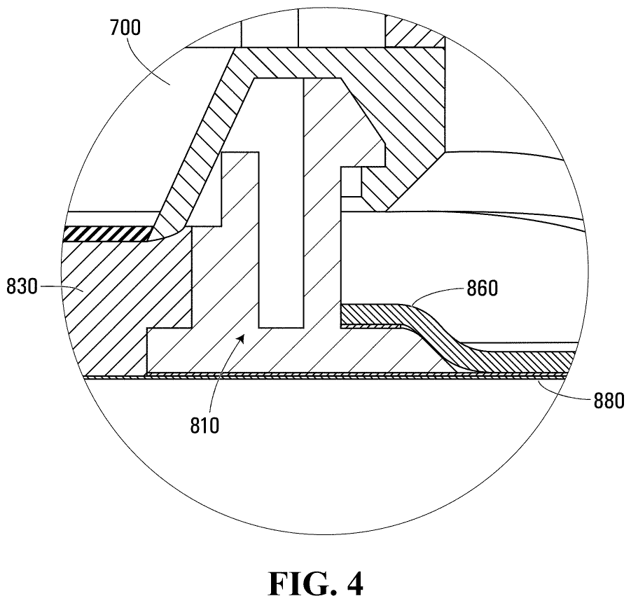

Referring now to FIG. 4, shown is a detailed view of a mechanism of the monitoring pad 800 for receiving and holding the ultrasound probe 700. In some implementations, the ultrasound probe 700 clips into the cradle 810 with application of manual pressure. In the illustrated example, a protruding portion of the cradle 810 penetrates into the ultrasound probe 700, and a hook portion of the protruding portion secures into a corresponding recess in the ultrasound probe 700. However, it is to be understood that this is a very specific way to receive and hold the ultrasound probe 700 and that other implementations are possible and are within the scope of this disclosure.

There are many possible materials for the support structure 810,840, 850,860. In specific implementations, the backing layer 860 is a foam backing layer formed of polyurethane, the clip 840 is a silicon retaining structure, and the cradle 810 is a retaining structure formed of silicon or a polymer. However, other implementations are possible. For example, metal, composite, carbon and elastomer materials are materials that can be used for the support structure 810,840,850,860 of the monitoring pad. In some implementations, a rigid material (e.g. metal, carbon) is used for the cradle 810 and the clip 840, but not for the support layer 860,850. In some implementations, the components 810,840,850,860 are bonded together. For example, in some implementations, the cradle 810 is bonded to the backing layer 860 via the adhesive layer 815. However, any suitable way of combining the components 810,840,860 can be employed. In another implementation, the support structure 810,840,860 is a single material and not a combination of different components.

In some implementations, the support layer 860,850 of the support structure 810,840,850,860 is not disposed in a region underneath the ultrasound gel pad 830. Rather, the support layer 860,850 generally surrounds the ultrasound gel pad 830. In this way, during an ultrasound procedure, ultrasound pulses and the resulting reflections do not need to traverse the support layer 860,850. This can enable direct contact between the ultrasound gel pad 830 and the patient. In other implementations, at least a portion of the support layer 860,850, for example the backing layer 860, is disposed underneath the ultrasound gel pad 830. This can help to contain the ultrasound gel pad 830. For such implementations, the backing layer 860 can be a thin polyurethane layer to enable ultrasound beams to pass through.

When the ultrasound gel pad 830 is said to be “sandwiched between the patient and the ultrasound probe 700”, it is to be understood that the ultrasound gel pad 830 is disposed between the patient and the ultrasound probe 700, generally with pressure being applied, even though it is possible that there is no direct contact between the patient and the ultrasound gel pad 830. It is possible that there is no direct contact between the patient and the ultrasound gel pad 830 due to one or more intervening layers, such as the backing layer 860 and/or an adhesive layer 880. However, direct contact between the patient and the ultrasound gel pad 830 can improve the ultrasound interface. Hence, direct contact is provided for the implementations that are depicted herein. Similarly, it is possible that there may be no direct contact between the ultrasound probe 700 and the ultrasound gel pad 830 due to one or more intervening layers, such as a coupling material 740. However, direct contact between the ultrasound probe 700 and the ultrasound gel pad 830 is certainly possible.

Although FIG. 3 and FIG. 4 depict a specific implementation for the support structure 810,840,850,860, it is to be understood that other support structures are possible and are within the scope of the disclosure. Components such as the cradle 810, the support layer 860,850, and the clip 840 are very specific and are provided merely as an example. In another implementation, a support structure (not shown) includes straps or bands to hold the ultrasound probe 700 in the fixed arrangement against the ultrasound gel pad 830. More generally, any suitable support structure that can receive and hold the ultrasound probe 700 in the fixed arrangement against the ultrasound gel pad 830 can be implemented. Other implementations could include for example magnetic fixation systems (not shown) or any other mechanical designs (not shown) that can fix the ultrasound probe 700 onto the monitoring pad 800. Other implementations are possible.

There are many possibilities for the ultrasound gel pad 830. In some implementations, the ultrasound gel pad 830 is a solid ultrasound gel acting as a coupling material between the patient and the ultrasound transducers of the ultrasound probe 700. In some implementations, the ultrasound gel pad 830 mechanically acts as an impedance matcher for the ultrasound transducers. In some implementations, the thickness of the ultrasound gel pad 830 is designed so that the ultrasound probe 700 can make suitable contact with it. In some implementations, the ultrasound gel pad 830 is provided with a removable layer 820. The removable layer 820 acts as a protector to the ultrasound gel pad 830 to help ensure that the ultrasound gel pad 830 remains viable before the monitoring pad 800 is used. The removable layer 820 can be removed (i.e. peeled off) before attaching the ultrasound probe 700. In other implementations, the monitoring pad 800 has no such removable layer 820.

In some implementations, the monitoring pad 800 has an adhesive layer 880 for securing the monitoring pad 800 to the patient. In some implementations, the adhesive layer 880 is geometrically shaped to correspond with the support layer 860,850 of the support structure 810,840,850,860, and more specifically the backing layer 860. In some implementations, the adhesive layer 880 includes an acrylate material. In some implementations, the adhesive layer 880 has chemical and mechanical properties to resist normal shear and tear forces when applied on a prepared and cleaned surface of the patient. In some implementations, at least the backing layer 860 and the adhesive layer 880 are made of biocompatible material, and the adhesive layer 880 is made of material that promote adhesion to skin and prevents adverse skin reaction.

In some implementations, the monitoring pad 800 has a removable layer 890 covering the adhesive layer 880. In some implementations, the removable layer 890 has two parts (i.e. a first part and a second part) that are referred to as “liners”. The removable layer 890 acts as a protector to the adhesive layer 880 to help ensure that the adhesive layer 880 remains viable before the monitoring pad 800 is used. In some implementations, the ultrasound gel pad 830 is held in place by the removable layer 890. The removable layer 890 can be removed (i.e. peeled off) before applying the monitoring pad 800 to the patient. In other implementations, the monitoring pad 800 has no such removable layer 890.

Although the monitoring pad 800 is shown with the adhesive layer 880 and the removable layer 890, it is noted that other implementations are possible in which there is no adhesive layer 880 and no removable layer 890. Other means for securing the monitoring pad 800 to the patient are possible and are within the scope of the disclosure. For example, in another implementation, straps or bands are used to secure the monitoring pad 800 to the patient instead of the adhesive layer 880.

In some implementations, the monitoring pad 800 has at least one sensor 870 unrelated to ultrasound. This can enable acquisition of additional data that may supplement an ultrasonic image. There are many possibilities for the sensor 870. In some implementations, the sensor 870 includes a pair of electrocardiogram electrodes 870 for sensing a heartbeat. In specific implementations, as shown in the illustrated example, the monitoring pad 800 has a copper layer 870 or any suitable alternative (e.g. aluminum layer) wherein this layer has sensor devices like electrocardiogram electrodes and routing wire for connectivity and signal transmission. In specific implementations, the electrocardiogram electrodes 870 are dry electrodes made via a printed electronic process using, for example, carbon and silver/silver chloride (Ag/AgCl) inks, although wet (gel) electrodes are possible as well. Additionally, or alternatively, the sensor 870 can include a blood oxygen saturation sensor for sensing a blood oxygen saturation. Other implementations are possible. More generally, any suitable sensor or set of sensors unrelated to ultrasound can be implemented.

In some implementations, for each sensor 870 unrelated to ultrasound, the monitoring pad 800 has wiring, cabling and/or connectors 875 from the sensor 870 to the ultrasound probe 700. This can enable acquisition of the additional data for the ultrasound beamforming device 900 via the ultrasound probe 700 and the cable 600. In some implementations, the ultrasound probe 700 has wiring, cabling and/or connectors to provide sensor signal to the ultrasound beamforming device 900. In some implementations, the cable 600 includes wiring for the ultrasound transducers and separate wiring for the sensor 870 unrelated to ultrasound. Other implementations are possible.

In some implementations, the ultrasound probe 700 includes a bottom case 710 and an upper case 720 as illustrated, although other configurations are possible. An ultrasound transducer array (not shown) would be disposed within the bottom case 710 of the ultrasound probe 700, such that the ultrasound transducer array can make contact with the ultrasound gel pad 830 through an opening of the bottom case 710 when the ultrasound probe 700 is connected to the monitoring pad 800. In some implementations, the ultrasound probe 700 also has a strain relief 730 to support the cable 600 that is connected to the ultrasound probe 700. The cable 600 can include wiring for the ultrasound transducer array and/or the other sensor 870. The strain relief 730 can help to prevent the cable 600 and its wiring therein from being accidentally pulled out of the ultrasound probe 700.

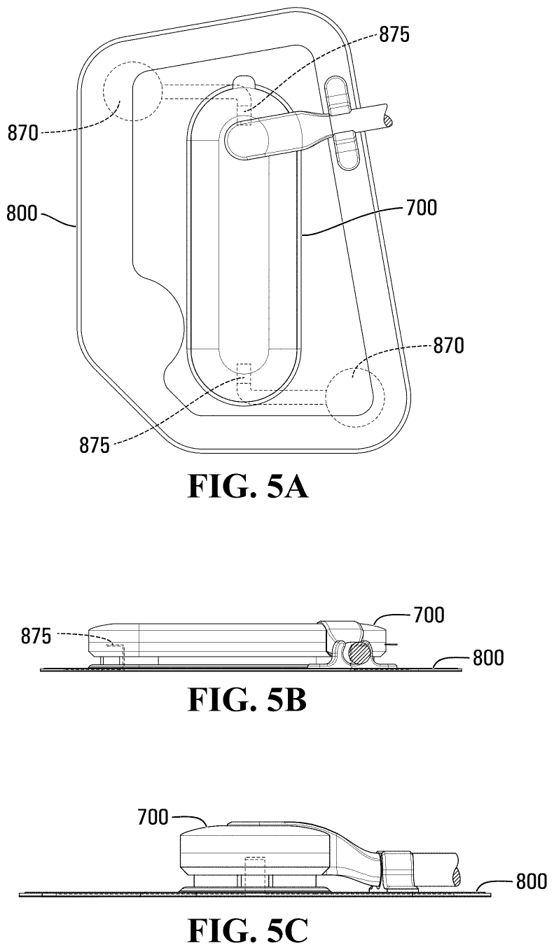

Referring now to FIG. 5A to FIG. 5C, shown are schematics depicting the ultrasound probe 700 connected to the monitoring pad 800. FIG. 5A is a schematic of a top view, while FIG. 5B and FIG. 5C are schematics of side views. As shown, the connectors 875 for the sensor 870 are embedded in the cradle 810 and connect to the ultrasound probe 700 when the ultrasound probe 700 is fixed on the cradle 810. In some implementations, the monitoring pad 800 has a pictogram (not shown) for position indication and guidance, and/or guidance and locations of the sensor 870. The pictogram can appear on any suitable surface, for example the support layer 860 of the support structure 810,840,850,860. More specifically, the pictogram can appear on the frame 850 of the support layer 860,850. Other implementations are possible.

In some implementations, the ultrasound system 100 has lights (not shown) on or near the monitoring pad 800 to provide visual feedback to an operator. The lights could include LEDs (Light Emitting Diodes) incorporated in the monitoring pad 800 and/or the ultrasound probe 700 (including for example the strain relief 730 of the ultrasound probe 700) to light up the cradle 810, the ultrasound probe 700 or the cable 600, for example. The lights could be used for signalling the operator a status of the ultrasound system, for example that the ultrasound system 100 is operational, a signal(s) has been detected, and/or there is a malfunction in the ultrasound system 100.

### Ultrasound Transducer Array

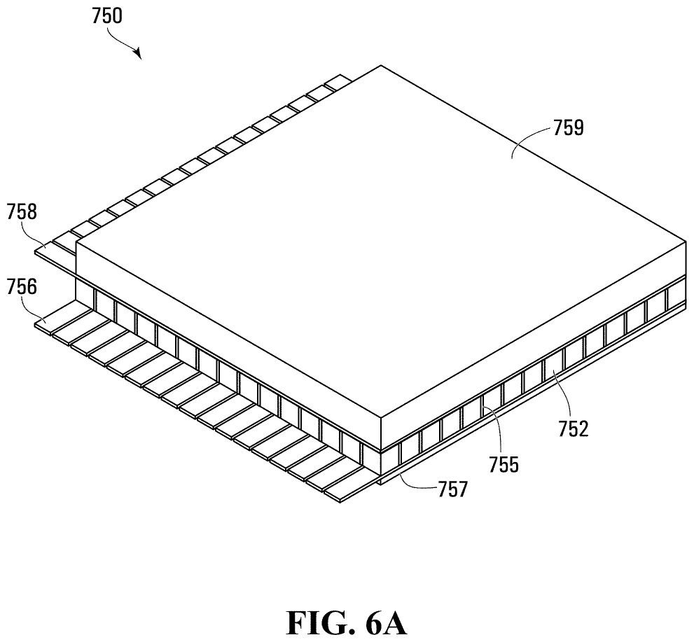

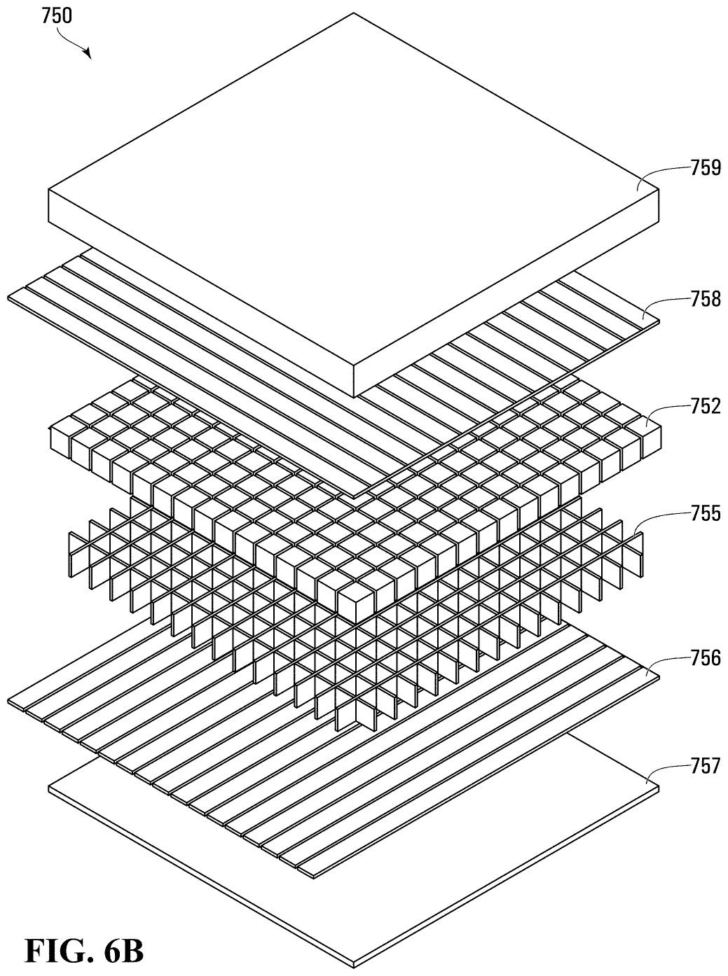

Referring now to FIG. 6A and FIG. 6B, shown are schematics of an ultrasound transducer array 750 of the ultrasound probe 700. FIG. 6A shows an assembled view of the ultrasound transducer array 750, while FIG. 6B shows an exploded view of the ultrasound transducer array 750. The ultrasound transducer array 750 is a main component of the ultrasound probe 700, which can be connected to the monitoring pad 800 for a medical ultrasound procedure as described above. The ultrasound transducer array 750 is operatively coupled to the monitoring pad 800 for ultrasound beam emission and reception. When they are assembled together they constitute a “hands-free ultrasound probe”, and can be used with the ultrasound beamforming device 900 for signal processing and real-time imaging. The assembly of the hands-free ultrasound probe with the ultrasound beamforming device 900 constitutes an ultrasound system that can be used for imaging and monitoring purposes.

The ultrasound transducer array 750 has an array of piezo-electric elements 752. In some implementations, the piezo-electric elements 752 are PMUT (Piezoelectric Micromachined Ultrasonic Transducers), which are a MEMS (Microelectromechanical Systems) based piezoelectric ultrasonic transducer. In other implementations, the ultrasound transducer array 750 has piezoelectric alternatives like electrostrictive material, or alternatively PMUT or CMUT (Capacitive Micro-machined Ultrasound Transducer) materials.

In some implementations, the piezo-electric elements 752 are geometrically arranged between a top electrode array and a bottom electrode array for piezoelectric voltage/current excitation. In particular, the piezo-electric elements 752 have top electrodes 758 and bottom electrodes 756 that are disposed orthogonally as illustrated, although other implementations in which an angular positions other than 90 degrees are possible. Voltage application with electrical pulses to the top electrodes 758 and the bottom electrodes 756 of the piezo-electric elements 752 causes the piezo-electric elements 752 to emit ultrasound energy.

In some implementations, the piezo-electric elements 752 are embedded within a composite matrix 755. In some implementations, the composite matrix 755 is a polymer composite material that can include polytetrafluoroethylene or PVDF (polyvinylidene fluoride), for example.

In some implementations, the ultrasound probe 700 also has a matching layer 757, which can be in silicon or sol-gel SiO2/polymer nano-composites, for example, and a damping block 759, which can be in tungsten loaded araldite (epoxy), for example. The matching layer 757 is used to improve the efficiency of energy transfer into and out of a patient and the damping block 759 absorbs the backward directed ultrasound energy and attenuates stray ultrasound signals.

In some implementations, the ultrasound transducer array 750 has M×N ultrasound elements 752, where M and N are natural numbers, forming the largest array aperture of the transducer. In other words, the ultrasound transducers 752 are oriented in a two-dimensional array. In some implementations, the ultrasound transducer array 750 has a (M×N)² number of minimal apertures, where a minimal aperture has at least two elements. An aperture is an active area that transmits or receives acoustic wave at certain moment. In the illustrated example, the ultrasound transducer array 750 is rectangular in shape. However, other two-dimensional shapes are possible, such as a circular shape or an oval shape for example.

In some implementations, the ultrasound beamforming device 900 is configured to utilize one array of the two-dimensional array as a single linear array. In other implementations, the ultrasound transducers 750 has a linear array of M ultrasound elements, where M is a natural number forming the largest linear aperture of the transducer. Thus, it is to be understood that an “ultrasound transducer array” does not need to be a two-dimensional array. In some implementations, the ultrasound transducer array 750 has a M² number of minimal apertures, where a minimal aperture has at least two elements. An aperture is an active area that transmits or receives acoustic wave at certain moment.

In some implementations, the ultrasound elements 752 can be selected using the total aperture of the ultrasound elements 752 or can be selected individually for creating a sub-aperture. Using full aperture or sub-aperture, emission and reception of the ultrasound beam can be configured individually in order to adjust time-delay of each elements of the array for providing path length of ultrasound beam propagation. Time-delay corrections is a method where a phase control is applied to individual acoustic beam allowing both angular ultrasound beam steering in azimuth and elevation directivity and allowing also depth focusing.

In some implementations, the ultrasound transducer array 750 uses time-delay phased array or alternative beamforming techniques for automatically adjusting an ultrasound beam to be focused in a 3D inspected volume by providing methods for steering in two orthogonal angles: the azimuth and the elevation angles. In some implementations, ultrasound beamforming techniques enable depth and directivity of ultrasound beam for image contrast enhancement and pattern recognition for diagnostic purpose.

In some implementations, the ultrasound transducer array 750 provides emission and reception of acoustic ultrasound beams in media and where emission and reception of ultrasound beams in media are controlled and monitored using signal and imaging processing techniques implemented by the ultrasound beamforming device 900. In some implementations, signal processing in the ultrasound beamforming device 900 provides volume angular scanning with automatic depth and gain adjustment features for improving signal to noise ratio.

In some implementations, the ultrasound transducer array 750 is geometrically configured in a way that streamlines a fixation process to the monitoring pad 800. Traditional ultrasound transducers are vertically designed in order to handle a probe for body pressure and rotation, enabling 3D angular rotation of the probe for geometry positioning and focusing. By contrast, the hands-free ultrasound probe has a surface design array of elements that are geometrically dimensioned and spaced between them to enable 3D angular steering of ultrasonic beams in the volume of inspection.

The ultrasound transducer array 750 is oriented within the ultrasound probe 700 such that the ultrasound transducer array 750 is substantially parallel to a surface of the patient. In some implementations, the ultrasound transducer array 750 is oriented at an angle of 0° with a long axis of the ultrasound probe 700. In other implementations, the ultrasound transducer array 750 is oriented at an angle different from 0° to the long axis of the ultrasound probe 700, for example 30°, in order to geometrically facilitate beam focusing to an area of interest, thus facilitating for example an acquisition of a parasternal long axis plane of cut of the heart. In some implementations, the angle of the ultrasound transducer array 750 can be manipulated or adjusted by a motor (not shown) within the ultrasound probe 700 to facilitate beam focusing to an area of interest. In other implementations, the angle can be manually manipulated or adjusted. In other implementations, the angle remains fixed. Other implementations are possible and are within the scope of the disclosure.

Further example details of how the ultrasound transducer array 750 can be operated by the ultrasound beamforming device 900 are provided below with reference to FIG. 7.

### Ultrasound Beamforming Device

Referring now to FIG. 7, shown is a block diagram of the ultrasound beamforming device 900 operatively coupled to the ultrasound transducer array 750 and another sensor 870 unrelated to ultrasound. It is to be understood at the outset that the ultrasound beamforming device 900 is shown with a very specific combination of components, and that other combination of components are possible. The assembly of the ultrasound probe 700 (having the ultrasound transducer array 750 and the other sensor 870) with the ultrasound beamforming device 900 constitutes an ultrasound system that can be used for imaging and monitoring purposes.

The ultrasound beamforming device 900 has control hardware 200 for controlling transmission and reception over the ultrasound transducer array 750, data acquisition and signal processing electronics 400 for processing received data, processing hardware 300 for processing and displaying the data, and a bus 500 for enabling interactivity. In some implementations, the control hardware 200 has a plurality of control channels for signal processing as described below.

In some implementations, the control hardware 200 has components for transmission over the ultrasound transducer array 750, including a Tx (Transmitting) FPGA (Field Programmable Gate Array) beamformer 240 and a CW (Continuous Wave) transmitter 210. In some implementations, the control hardware 200 also has components for reception over the ultrasound transducer array 750, including an Rx (Receiving) FPGA beamformer 260. In some implementations, the control hardware 200 also has a signal conditioning unit 280 for interacting with the sensor 870. In some implementations, an HV (High Voltage) control switch Tx/Rx 230 and HV multiplexers 270 select between a transmission mode and a reception mode, for example based on control from the Tx FPGA beamformer 240.

In some implementations, the control hardware 200 is configured to selectively apply a bias voltage to a set of planar electrodes for performing apodization and aperture selection. The bias voltage can include multiple levels of positive, negative or zero bias voltages from the bias voltage generator 220. The selective application of the bias voltage is performed by the HV control switch Tx/Rx 230 via high voltage multiplexers 270.

The control hardware 200 can cycle between the transmission mode and the reception mode for a medical ultrasound procedure. During the transmission mode, the HV multiplexers 270 enable transmission of a continuous wave signal from the CW transmitter 210, for example based on control from the Tx FPGA beamformer 240. Based on the apodization and aperture selection, the transmission over the ultrasound transducer array 750 is focused on a focal point in space. During the reception mode, the HV multiplexers 270 enable reception of signals over the ultrasound transducer array 750, based on resulting reflections from within the patient. The Rx FPGA beamformer 260 receives these signals via the control switch Tx/Rx 230.

In some implementations, the control hardware 200 has an FPGA Master 250 that functions as a delay controller by controlling application of the bias voltages from the bias voltage generator 220. In this way, the FPGA Master 250 can control the bias voltages across each respective set of planar electrodes of the ultrasound transducer array 750 to control a length of each respective variable delay. In some implementations, determining levels of positive, negative or zero bias voltage by the bias voltage generator 220, determining waveform signals generated by the CW transmitter 210, and selectively applying the same to a set of planar electrodes is sufficient to generate ultrasound energy in a space wherein an ultrasound focal point can be generated. Likewise, in some implementations, determining levels of positive, negative or zero bias voltage by the bias voltage generator 220, and selectively applying the same to a set of planar electrodes is sufficient to enable material transduction of an acoustic beam energy generated by a time-delayed ultrasound echo in space.

In some implementations, the ultrasound pulse is transmitted to the ultrasound focal point according to a specific focal law, and at least two planar electrodes of the ultrasound transducer array 750 can constitute a minimal set of planar electrodes as described above. In some implementations, each variable delay applied by a bias voltage across each respective set of planar electrodes generates an ultrasound pulse that is specific to a focal point and specific to a focal law. In some implementations, by grouping a set of multiple delays that each refer to an individual focal law, multiple other focal laws are applicable. In some implementations, the use of focal laws to control time-delay of each respective set of planar electrodes generates a plural set of ultrasound beam that are transmitted into a volume where the ultrasound energy may be in phase to a predefined focal point, wherein the focal point may provide depth and angular beam steering directivity in azimuth and elevation angles, respectively.

In some implementations, a bias voltage is applied across each respective set of planar electrodes such that an ultrasound echo can be received operationally coupled to a specific focal law. In some implementations, each variable delay applied to the received signal from the set of planar electrodes by the processing of a bias voltage across each respective set of planar electrodes enables material acoustic energy transduction of ultrasound echoes and wherein the control and processing of time-delay to received signal operationally refers to a specific focal law. In some implementations, by grouping a set of multiple delays that each refer to an individual focal law, a set of focal laws are applicable, and wherein focal laws generated for the ultrasound transmitting operation can, without limitation, inversely be used as time reversed focal laws for receiving operations. In some implementations, the use of focal laws to control the time-delay of each respective set of planar electrodes in a way such as to adjust the phase of the acoustic energy to a focal point in space, wherein the focal point may provide depth and angular beam steering directivity in azimuth and elevation angles respectively in a reception operation.

In some implementations, the FPGA Master 250, the Tx FPGA beamformer 240, and the Rx FPGA beamformer 260 are part of the same FPGA. However, other implementations are possible in which separate FPGAs are utilized. Also, other implementations are possible in which a DSP (Digital Signal Processor), microcontrollers, or other suitable hardware components are utilized instead of, or in addition to, an FPGA. More generally, the ultrasound beamforming device 900 can be implemented with hardware, software, firmware, or any suitable combination thereof.

In some implementations, the data acquisition and signal processing electronics 400 has a memory 410 for signal acquisition buffering, and an image & monitoring processor 420. In some implementations, the image & monitoring processor 420 is provided for both sensing and actuating the ultrasound transducer array 750, and for processing measured signals in order to compute and to improve image reconstruction. In some implementations, the image & monitoring processor 420 enables methods, procedures and algorithms for generation and reception of ultrasound wave signals, which can include standard phased array techniques based on time-delay and waveform generator algorithms or any other alternative time-delay beamforming methods without limitation transducers array patterns matching with said beamforming methods and algorithms to dynamically improve acoustic emission ultrasound beam energy and acoustic reception of said ultrasound beam echoes, namely methods and algorithms for improving signal to noise ratio.

In some implementations, the processing hardware 300 has a processor 320 configured to define voltage levels with the bias voltage generator 220 and waveform signals generated via the Tx FPGA beamformer 240 and the CW transmitter 210 to the set of planar electrodes to achieve an ultrasound focal point in space, during the transmission mode. In some implementations, the processor 320 is also configured to define the voltage levels to select from the bias voltage generator 220 for the set of planar electrodes to receive the acoustic beam energy generated by an ultrasound echo in space, during the reception mode. In some implementations, the processing hardware 300 has a GPU (Graphics Processing Unit) 330 for generating an ultrasonic image based on the reception of ultrasound wave signals, and wherein the GPU 330 can integrate processing features of the image & monitoring processor 420 and the processor 320, and a monitor/display 340 for displaying the ultrasonic image. In some implementations, the processing hardware 300 also has various peripherals 310 such as PCIe (Peripheral Component Interconnect express), USB (Universal Serial Bus) and Wifi, for example. Other implementations are possible.

In some implementations, the signal processing electronics 400 and/or the processing hardware 300 implement one or more algorithms. The one or more algorithms can include any one or appropriate combination of:

3D beam scanning algorithms, for example linear scan, sector scan, B-Mode and M-Mode imaging techniques for interrogating the volume of inspection;

3D beam scanning techniques such as Full Matrix Capture and Total Focusing Methods for interrogating the volume of inspection which can be used to improve signal to noise ratio and image reconstruction;

image processing algorithms enabling the reconstruction of an ultrasound image with the use of the 3D beam scanning algorithms;

segmentation and image pattern recognition algorithms for the identification of objects in images;

algorithms for reprogramming focal laws in order to refocus ultrasound beams at a specific ROI (Region of Interest), wherein the ROI may refer to a specific POI (Point of Interest) or a specific AOI (Area of Interest), and wherein refocusing of ultrasound beams improves signal to noise ratio;

signal processing algorithms, for example FFT (Fast Fourier Transform), convolution, transfer function computation of the set of planar electrodes referring to a pair of timely actuator/sensor combination from the emission and reception operations; and

algorithms for comparing the computed transfer function magnitude and phase spectrum for each actuator/sensor, wherein the computed transfer function magnitude and phase spectrum include algorithms for identifying ultrasound energy distribution of a set of actuator/sensor paring wherein the spectrum information on magnitude and phase comprise frequency selection and shifting of signal waveform generation and time-delay techniques for refocusing ultrasound energy in a region of interest in the interrogated volume.

In some implementations, as depicted in FIG. 7, the ultrasound beamforming device 900 is configured to receive a reading from the sensor 870 using the signal conditioning unit 280. In some implementations, the ultrasound beamforming device 900 is configured to receive the reading via the ultrasound probe 700, for example through the cable 600 or by other means, when the sensor 870 is connected to the ultrasound probe 700 via the connectors 875. In some implementations, signal conditioning circuit boards and multiplexing circuits are used to condition and multiplex signals to the beamforming device 900 via the cable 600. In some implementations, the ultrasound beamforming device 900 has a separate signalling path (not shown) other than the cable 600 for receiving the reading from the sensor 870.

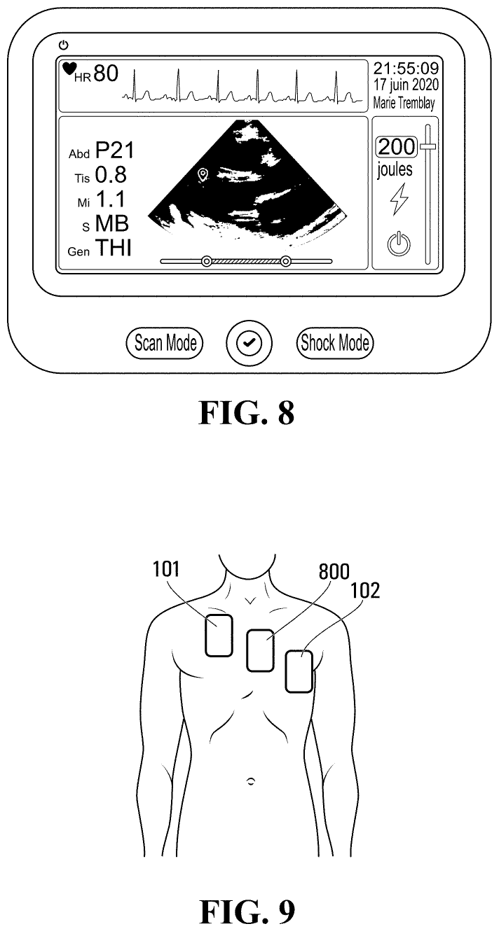

In accordance with an embodiment of the disclosure, the ultrasound beamforming device 900 concurrently displays an ultrasound image and another image based on the reading from the sensor 870. For example, FIG. 8 shows an ultrasound image being displayed concurrently with an electrocardiogram for a case of the sensor 870 being a pair of electrocardiogram electrodes 870 for sensing a heartbeat. Other displays are possible depending on the sensor 870. For example, in the case of the sensor 870 being a blood oxygen saturation sensor, the ultrasound beamforming device 900 may concurrently display an ultrasound image and a graph representing blood oxygen saturation over time. Other implementations are possible.

In some implementations, the ultrasound beamforming device 900 is configured to connect to defibrillator equipment and to control the defibrillator equipment and/or display information of the defibrillator equipment. For example, FIG. 8 shows an ultrasound image being displayed concurrently with an electrocardiogram from the defibrillator equipment. Also, FIG. 8 shows information of the defibrillator equipment (e.g. 200 joules, etc.) and provides controls for delivering an electric shock via the defibrillator equipment.

In other implementations, the ultrasound system 100 includes a full defibrillation system (e.g. defibrillation circuitry embedded into the beamforming device 900) and connected to two independent defibrillator electrodes in addition of the ultrasound probe 700 and monitoring pad 800. This implementation of the ultrasound system 100 can provide both ultrasound monitoring and defibrillation capacities. As people in the art will appreciate, such system can allow a reduction in time to diagnosis and intervention, as well as increased diagnostic accuracy in critical care situations.

In order to enable the ultrasound image to be generated by the ultrasound system 100 for a patient at the same time, or immediately after delivering an electric shock to the patient via the defibrillator equipment, the ultrasound system 100 is configured to be resilient to electric shocks from defibrillation. For instance, the ultrasound probe 700 and/or the ultrasound beamforming device 900 can be designed to have an input impedance high enough to avoid damage that may otherwise be caused by the electric shock, but also low enough to permit proper operation of the ultrasound system 100. Another means to render the ultrasound probe 700 resilient to electric shock may include a bypass circuit equivalent to an electrical switch that may avoid current/voltage damage caused by electrical shock. The monitoring pad 800 can be made of materials to be resilient as well.

In some implementations, for sensor integration, there is provided a means for protecting against a defibrillator pulse. That protection circuit can have a dual function of protecting the patient (e.g. by ensuring that the defibrillation pulse indeed goes through the patient and is not lost within the ultrasound beamforming device 900) and protecting the operator (e.g. by ensuring that the ultrasound beamforming device 900 remains safe for the operator even during defibrillation). If the ultrasound beamforming device 900 does not have an electrical contact to the patient, there may not be any need for such protection. However, in some implementations having the additional sensor 870 for an ECG signal, the ECG and ultrasound signals can be routed through separate electrical connectors within the cable 600.

Referring now to FIG. 9, shown is a schematic of a patient showing example placement of the monitoring pad 800 between a pair of defibrillation pads 101,102. In some implementations, the ultrasound system 100 (including the monitoring pad 800 and the ultrasound probe 700) is resilient to electric shocks from defibrillation as described above. Although the ultrasound system 100 is configured to be resilient to electric shocks from defibrillation, it is noted that the ultrasound system 100 does not have to be able to generate an ultrasound image simultaneously with defibrillation.

In some implementations, the ultrasound beamforming device 900 implements pattern recognition or artificial intelligence to automatically generate morphology or tissue identification (e.g. a specific plane of cut to help with a physician diagnosis) based on a combination of the resulting reflections of the ultrasound beam and the reading from the other sensor 870. As a specific example, a multi-layer artificial neural network can be trained with training data to recognise patterns corresponding to target morphology or tissue identification, and then the multi-layer artificial neural network used to automatically generate a morphology or tissue identification for situations that are similar to those represented by the training data. By combining information from an ultrasound image with information unrelated to the ultrasound (e.g. electrocardiogram and/or blood oxygen saturation), it may be possible to streamline physician diagnosis.

### Method of Using Ultrasound System

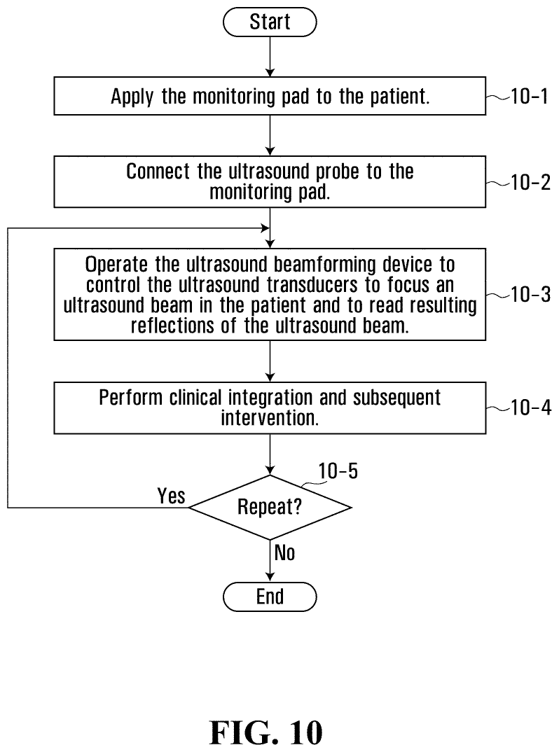

Referring now to FIG. 10, shown is a flowchart of a method of using the ultrasound system 100 for a medical ultrasound procedure. This method can be implemented by a user, for example by a technician, a nurse, a physician, or paramedic.

At step 10-1, the user applies the monitoring pad 800 to a patient. As described earlier, the monitoring pad 800 has an ultrasound gel pad 830 and a support structure 810,840,850,860 that holds the ultrasound gel pad 830. At step 10-2, the user connects the ultrasound probe 700 to the monitoring pad 800. As described earlier, the ultrasound probe 700 has an ultrasound transducer array 750.

In accordance with an embodiment of the disclosure, the support structure 810,840,850,860 is geometrically configured to receive the ultrasound probe 700 and to hold the ultrasound transducers in a fixed arrangement against the ultrasound gel pad 830, such that the ultrasound gel pad 830 is sandwiched between the patient and the ultrasound transducers.

At step 10-3, the user operates the ultrasound beamforming device 900 to control the ultrasound transducers to focus an ultrasound beam into the patient and to read resulting reflections of the ultrasound beam. In some implementations, the user operates the ultrasound beamforming device 900 without holding or manipulating the monitoring pad 800 or the ultrasound probe 700, which remain fixed to the patient. In some implementations, at step 10-3, the user performs clinical integration and subsequent intervention.

Steps 10-3 and 10-4 can be repeated as appropriate based on whether the user decides to continue at step 10-5. In some implementations, during the medical ultrasound procedure, the user performs a defibrillation process. Also, in some implementations, the user monitors heartbeat and/or blood oxygen saturation using the ultrasound system 100 through the sensors 870. Notably, the defibrillation process and the monitoring of the heartbeat and/or blood oxygen saturation can occur during the medical ultrasound procedure. Once the user decides to stop the medical ultrasound procedure at step 10-5, then the method ends.

### Other Embodiments

Another embodiment relates to volumetric ultrasound imaging in aid of defibrillation or monitoring procedure in critical care and in aid of multiplexed point-of-care diagnostics like electrocardiogram diagnostic as an example embodiment of this invention.

Another embodiment provides the use of a hands-free ultrasound transducer with a monitoring gel pad that includes electrocardiogram electrodes enabling ECG monitoring and features.

Another embodiment provides a combination of an imaging ultrasound system using a hands-free ultrasound transducer array and a monitoring pad comprising electrocardiogram electrodes in order to provide new monitoring features with the combination of ultrasound signal with ECG signal in a resuscitation context.

Another embodiment is a combination of an ultrasound imaging system using a hands-free ultrasound transducer array and a monitoring pad comprising electrocardiogram electrodes and a defibrillator circuit comprising electroshock electrodes in order to provide defibrillation in a resuscitation emergency context of a sick patient. For example, in some implementations, the monitoring pad 800 has defibrillation electrodes, such as metal-metal/chloride electrodes for example, that are multi-function electrodes that allow defibrillation, as well as conduct the electrical impulse generated by the heart and therefore provide information on the heart rate and precise cardiac rhythm, both useful information in resuscitation (see for example U.S. Pat. No. 5,080,099). In some implementations, the defibrillation electrodes provide an area of contact of 90 cm² around the transducers in compliance with guidelines for defibrillator pads, of 50 cm² per patch and a total 150 cm² with the body of a patient, for efficient defibrillation and decreased likelihood of inducing skin damage.

Another embodiment is a combination of ultrasound monitoring capacities with other forms of monitoring such as peripheral blood oxygen saturation.

Another embodiment includes post-acquisition image processing capacities allowing automated image recognition and data combination such as ECG (electrocardiography) and echography, for example.

Another embodiment includes echography generated without a clinicians involvement, for example by ambulance attendants or military personnel. Echography monitoring generates continuous data in a non-invasive way, with possible use of artificial intelligence.

Another embodiment provides a monitoring pad combined with other ultrasound components, to provide increased ultrasound diagnostic and monitoring capacities, such as automatized EGLS (Echo Guided Life Support) by pairing the heart with lung and variability or size of the IVC (Inferior Vena Cava), or a lung monitoring device for monitoring the presence of B-lines suggestive of water in the lungs for example.

Another embodiment is a transducer as described above that is adapted in shape and format to fit the neonatal and pediatric population or to fit other parts of the adult/pediatric body.

Numerous modifications and variations of the present disclosure are possible in light of the above teachings. It is therefore to be understood that within the scope of the appended claims, the disclosure may be practised otherwise than as specifically described herein.

We claim:

1. An ultrasound system comprising:
an ultrasound probe having a plurality of ultrasound transducers;
a monitoring pad for application to a patient, comprising:
an ultrasound gel pad; and
a support structure that holds the ultrasound gel pad and is geometrically configured to receive the ultrasound probe and to hold the ultrasound probe in a fixed position against the ultrasound gel pad, such that the ultrasound gel pad is sandwiched between the patient and the ultrasound probe; and
an ultrasound beamforming device configured to control the plurality of ultrasound transducers to focus an ultrasound beam into the patient and to read resulting reflections of the ultrasound beam.

2. The ultrasound system of claim 1, wherein:
the ultrasound beamforming device comprises transmission circuitry to control a time-delay for exciting each ultrasound transducer to generate a plurality of ultrasound beams transmitted into the patient such that ultrasound energy is in phase at a predefined focal point within the patient; and
the ultrasound beamforming device comprises reception circuitry to read resulting reflections of the ultrasound beam from the predefined focal point.

3. The ultrasound system of claim 1, wherein the ultrasound beamforming device is configured to refocus the plurality of ultrasound beams at a specific region of interest to improve signal to noise ratio.

4. The ultrasound system of claim 1, wherein the ultrasound transducers are oriented in a two-dimensional array.

5. The ultrasound system of claim 4, wherein the ultrasound beamforming device is configured to utilize one array of the two-dimensional array as a single linear array.

6. The ultrasound system of claim 1, wherein the ultrasound beamforming device comprises a display for displaying an ultrasound image based on the resulting reflections of the ultrasound beam.

7. The ultrasound system of claim 1, wherein the ultrasound beamforming device implements pattern recognition or artificial intelligence to automatically generate a morphology or tissue identification based on the resulting reflections of the ultrasound beam.

8. The ultrasound system of claim 1, wherein the monitoring pad comprises at least one sensor unrelated to ultrasound, and the ultrasound beamforming device is configured to receive a reading from the at least one sensor.

9. The ultrasound system of claim 8, wherein the ultrasound beamforming device is configured to receive the reading from the at least one sensor via the ultrasound probe.

10. The ultrasound system of claim 8, wherein the ultrasound beamforming device comprises a display for concurrently displaying an ultrasound image based on the resulting reflections of the ultrasound beam and another image based on the reading from the at least one sensor.

11. The ultrasound system of claim 8, wherein the ultrasound beamforming device implements pattern recognition or artificial intelligence to automatically generate a morphology or tissue identification based on a combination of the resulting reflections of the ultrasound beam and the reading from the at least one sensor.

12. The ultrasound system of claim 8, wherein the at least one sensor comprises a pair of electrocardiogram electrodes for sensing a heartbeat.

13. The ultrasound system of claim 8, wherein the at least one sensor comprises a blood oxygen saturation sensor for sensing a blood oxygen saturation.

14. The ultrasound system of claim 1, wherein:
the ultrasound beamforming device is configured to connect to defibrillator equipment and to control the defibrillator equipment and/or display information of the defibrillator equipment; and
the ultrasound system is resilient to electric shocks from defibrillation.

15. The ultrasound system of claim 1, wherein:
the ultrasound beamforming device comprises defibrillation circuitry; and
the ultrasound system is resilient to electric shocks from defibrillation.

16. The ultrasound system of claim 1, comprising:
LEDs (Light Emitting Diodes) or other lights disposed on the monitoring pad and/or the ultrasound probe for signalling a status of the ultrasound system.

17. A monitoring pad for application to a patient, comprising:
an ultrasound gel pad; and
a support structure that holds the ultrasound gel pad and is geometrically configured to receive an ultrasound probe and to hold the ultrasound probe in a fixed position against the ultrasound gel pad, such that the ultrasound gel pad is sandwiched between the ultrasound probe and the patient.

18. The monitoring pad of claim 17, wherein the support structure comprises:
a cradle that holds the ultrasound gel pad within a predefined boundary, and has a mechanism to receive the ultrasound probe and hold the ultrasound probe in the fixed position, such that the ultrasound gel pad is sandwiched between the ultrasound probe and the patient; and
a support layer for supporting the cradle to the monitoring pad.

19. The monitoring pad of claim 18, wherein the monitoring pad enables direct contact between the ultrasound gel pad and the patient.

20. The monitoring pad of claim 17, further comprising:
at least one sensor unrelated to ultrasound.

21. The monitoring pad of claim 20, further comprising:
for each sensor, a connector from the sensor to the ultrasound probe.

22. The monitoring pad of claim 20, wherein the at least one sensor comprises a pair of electrocardiogram electrodes for sensing a heartbeat.

23. The monitoring pad of claim 20, wherein the at least one sensor comprises a blood oxygen saturation sensor for sensing a blood oxygen saturation.

24. The monitoring pad of claim 17, wherein the monitoring pad is resilient to electric shocks from defibrillation.

25. An ultrasound beamforming device configured to control an ultrasound transducer array with beamforming to acquire ultrasound data, to receive a reading from at least one sensor unrelated to ultrasound, and to concurrently display an ultrasound image based on the ultrasound data and another image based on the reading from the at least one sensor.

26. The ultrasound beamforming device of claim 25, wherein the at least one sensor comprises a pair of electrocardiogram electrodes for sensing a heartbeat, and the image based on the reading from the at least one sensor comprises an electrocardiogram.

27. The ultrasound beamforming device of claim 25, wherein the at least one sensor comprises a blood oxygen saturation sensor for sensing a blood oxygen saturation, and the image based on the reading from the at least one sensor comprises a graph representing blood oxygen saturation over time.

28. The ultrasound beamforming device of claim 25, wherein the ultrasound beamforming device implements pattern recognition or artificial intelligence to automatically generate a morphology or tissue identification based on a combination of the ultrasound data and the reading from the at least one sensor.

29. A method comprising:
applying a monitoring pad to a patient, the monitoring pad having (i) an ultrasound gel pad and (ii) a support structure that holds the ultrasound gel pad and is geometrically configured to receive an ultrasound probe and to hold the ultrasound probe in a fixed position against the ultrasound gel pad;
connecting the ultrasound probe to the monitoring pad, the ultrasound probe having a plurality of ultrasound transducers; and
operating an ultrasound beamforming device to control the ultrasound transducers to focus an ultrasound beam into the patient and to read resulting reflections of the ultrasound beam.

30. The method of claim 29, comprising:
operating the ultrasound beamforming device without holding or manipulating the monitoring pad or the ultrasound probe.
